(19) United States
(12) Patent Application Publication
(10) Pub. No.: US 2004/0029773 A1
(43) Pub. Date: Feb. 12, 2004

(54) Use of 1-ebio in the treatment of bipolar disorders

(76) Inventors: Charles H. Large, Verona (IT); Georg Christian Terstappen, Verona (IT)

Correspondence Address: DAVID J LEVY, CORPORATE INTELLECTUAL PROPERTY, GLAXOSMITHKLINE, FIVE MOORE DR., PO BOX 13398, RESEARCH TRIANGLE PARK, NC, 27709-3398, US

(21) Appl. No.: 10/415,514

(22) PCT Filed: Nov. 2, 2001
(86) PCT No.: PCT/GB01/04871

(30) Foreign Application Priority Data
Nov. 2, 2000 (GB) 0026838.3

(51) Int. Cl. A61K 31/00; G01N 33/53; G01N 33/567

(52) U.S. Cl. 514/1; 435/7.2

(57) ABSTRACT

Pharmaceutical preparations and methods of treating a human afflicted with bipolar disorder, particularly the manic phase thereof. The preparations and methods involve 1-EBIO.

### FIELD OF THE INVENTION

The invention relates to openers of calcium activated potassium channels and their use in the treatment of bipolar disorder.

### BACKGROUND OF THE INVENTION

Calcium activated potassium (K⁺) channels play an important role in controlling the excitability and function of many cell types including neurones, epithelial cells, T-lymphocytes and skeletal muscle. Calcium actived K⁺ channels are activated by submicromolar increases in intracellular calcium and this activation is mediated by calmodulin. In excitable cells, activation of SK channels generates a hyperpolarising K⁺ current which contributes to the after-hyperpolarisation (AHP) that follows an action potential. This AHP modulates cell firing frequency and spike frequency adaptation thereby influencing neuronal excitability. SK channels have been implicated in diverse physiological functions such as learing and memory, synaptic enhancement and long-term potentiation.

Calcium activated potassium channels can be divided into three main classes. Large conductance (BK), intermediate conductance (IK) and small conductance (SK) calcium activated K⁺ channels. SK channels belong to the class of ligand-gated ion channels and to date three subytpes (SK-1, SK-2, SK-3) have been identified. The cloning of IK and SK-1, SK-2 and SK-3 channels has been described in WO 98/11139, the entire contents of which are incorporated herein by reference and to which the reader is specifically referred.

SK channels have a small single channel conductance for potassium of less than 25 pS and are voltage-independent. The bee venom peptide toxin apamin specifically blocks SK-2 and SK-3 channels and recently SK-1 channels have been found to be sensitive to apamin when expressed in HEK293 cells.

### SUMMARY OF INVENTION

The present invention provides the use of a SK-3 channel opener in the manufacture of a medicament for the treatment of bipolar disorder, particular in treatment of the manic phase of bipolar disorder.

The invention also provides a method for treating a patient afflicted with bipolar disorder, the said method comprising administering to a patient in need of treatment a therapeutically effective amount of an SK-3 channel opener.

### DETAILED DESCRIPTION OF THE INVENTION

The invention provides the use of a SK-3 channel opener in the manufacture of a medicament for the treatment of bipolar disorder, particularly the manic phase. A SK-3 channel opener is a compound which activates SK-3 channels. A SK-3 channel is activated when it opens to allow potassium ions to flow through the channel.

The SK-3 channel opener may activate IK, SK-1 and/or SK-2 channels in addition to SK-3 channels. Preferably the SK-3 channel opener selectively activates SK channels. A compound which selectively activates SK channels will activate SK channels but will not activate or will activate less strongly IK channels. More preferably the SK-3 channel opener selectively activates SK-3 channels. A compound which selectively activates SK-3 channels will activate SK-3 channels but will not activate or will activate only to a lesser extent SK-1 and SK-2 channels.

Preferably the SK-3 channel opener will maintain the calcium dependence of the SK-3 channel, i.e. the SK-3 channel opener will enhance the opening of SK-3 channels in response to an increase in calcium concentration but will not open SK-3 channels in the absence of calcium.

The SK-3 channel opener may enhance SK-3 channel activity directly by interacting with the SK-3 channel or indirectly by interacting with intracellular Ca²⁺ or calmodulin.

The SK-3 channel opening activity of a test agent may be assessed using any suitable test system. A typical test system comprises contacting cells expressing SK-3 channels with a test agent and monitoring activation of the channels using conventional electrophysiological methods such as patch clamping or conventional fluorescence methods such as DiBac4(3) membrane potential assays utilising a Fluorescence Image Plate Reader (FLIPR Molecular Devices). For example, recombinant SK-3 channels may be expressed in mammalian cell lines or in Xenopus oocytes and the effects of a test agent on channel opening may be monitored using standard electrophysiological techniques. Suitable mammalian cell lines include CHO, COS and HEK239 cells. The recombinant SK-3 channel may be expressed tranisently in a cell line. More preferably a stable recombinant cell line is used. Control experiements may be carried out using cells that do not express the recombinant SK-3 channel. The specificity of the observed potassium channel activity of a test agent may be assessed by monitoring activity in the presence of a SK-3 channel blocker such as apamin.

Binding of a test agent to a SK-3 channel may be monitored using a binding assay. For example, displacement of radiolabelled apamin by a test agent from cells, or cell membranes, expressing recombinant SK-3 indicates that an agent may act as a modulator of SK-3 channel activity. Further functional experiements are necessary to determine whether a test agent that binds SK-3 acts as an opener of SK-3 channels.

To test for the selectivity of a test agent for SK-3 channels the test agent may be tested first on cells expressing recombinant SK-3 channels, then on cells expressing recombinant IK channels and/or recombinant SK-1 and/or SK-2 channels. A test agent may be considered to be selective for SK-3 channels if the potassium channel activity observed on application of the test agent to cells expressing SK-3 channels is 2 times larger, preferably 3, 4, 5, 6 or 10 times greater, more preferably 15 or 20 fold greater than the potassium channel activity obtained on application of the test agent to cells expressing other subtypes of calcium activated potassium channels. Potassium channel activity may be determined using any suitable parameter, for example the threshold concentration at which the test agent activates a potassium channel.

A SK-3 channel opener may thus be identified by:

(i) expressing a recombinant SK-3 channel in a cell;

(ii) incubating said cell in the presence of a test agent; and

(iii) monitoring SK-3 channel activity thereby determining whether the test agent acts as an opener of SK-3 channels;

(iv) optionally comparing the activity determined in step (iii) with SK-1 and/or SK-2 channel opening activity following incubation of a cell, preferably said cell, in the presence of said test thereby determining the selectivity of said test agent for SK3 channels.

In another aspect of the present invention there is provided a method of identifying a lead series of candidate agents for the treatment of bipolar disorder, particularly the manic phase thereof, which method comprises the steps of;

(i) expressing a recombinant SK-3 channel in a cell;

(ii) incubating said cell in the presence of a test agent; and

(iii) monitoring SK-3 channel activity thereby determining whether the test agent acts as an opener of SK-3 channels;

(iv) optionally comparing the activity determined in step (iii) with SK-1 and/or SK-2 channel opening activity following incubation of a cell, preferably said cell, in the presence of said test agent thereby determining the selectivity of said test agent for SK3 channels.

The term “lead series” is a term of the art and refers in this instance to a plurality of candidate agents as defined infra. Aptly, a single member or sub-grouping of this plurality is selected for optimisation typically by chemical structural modification, of various characteristics such as solubility, pharmacokinetics etc and then further developed into a regulatory permissble medicament, typically a pharmaceutical preparation. The chemically modified version of course retains SK3 channel opening ability. The lead series will usually consist of less than 20 candidate agents, more typical less than 10, e.g. 5 or less.

The term “test agent” denotes a compound whose capability as a SK3 channel opener has not yet been determined. The term “candidate agent” denotes a compound whose SK3 channel opening capability is known or assumed or has been identified as having such an ability by the method described supra.

In accordance with another aspect of the present invention there is provided a method of developing a SK3 channel opener suitable for administration to a human patient afflicted with bipolar disorder, particularly for the manic phase thereof, which method comprises;

(i) expressing a recombinant SK-3 channel in a cell;

(ii) incubating said cell in the presence of a test agent;

(iii) assessing SK-3 channel activity;

thereby determining whether the test agent acts as an opener of SK-3 channels;

(iv) optionally comparing the activity determined in step (iii) with SK-1 and/or SK-2 channel opening activity following incubation of a cell, preferably said cell, in the presence of said test agent thereby determining the selectivity of said test agent for SK3 channels;

(v) providing a chemically modified version of said agent of step (iii) or (iv)

(vi) administrating said version of step (v) to a human volunteer afflicted with bipolar disorder;

(vii) assessing whether the condition and/or symptoms of said volunteer of step (vi) improves.

Improvement of symptoms is intended to be understood that the clinically adverse symptoms of said patient are alleviated to a clinically significant extent. This maybe determined either by observing a reduction in the intensity and/or duration of symptoms. A SK3 channel opener is suitable for administration to a human if it has, inter alia, acceptable efficacy, pharmacokinetic and side effect profiles.

In another aspect there is provided a method of developing a SK3 channel opener suitable for administration to a human patient afflicted with bipolar disorder, particularly for the manic phase thereof, which method comprises;

(i) expressing a recombinant SK-3 channel in a cell;

(ii) incubating said cell in the presence of a test agent;

(iii) assessing SK-3 channel activity;

thereby determining whether the test agent acts as an opener of SK-3 channels;

(iv) optionally comparing the activity determined in step (iii) with SK-1 and/or SK-2 channel opening activity following incubation of a cell, preferably said cell, in the presence of said test agent thereby determining the selectivity of said test agent for SK3 channels;

(v) providing a chemically modified version of said agent of step (iii) or (iv).

(vi) Using said version of step (v) in the manufacture of a medicament.

Also provided is a method of developing a SK3 channel opener suitable for administration to a human patient afflicted with bipolar disorder, particularly for the manic phase thereof, which method comprises;

(i) expressing a recombinant SK-3 channel in a cell;

(ii) incubating said cell in the presence of a test agent;

(iii) assessing SK-3 channel activity;

thereby determining whether the test agent acts as an opener of SK-3 channels;

(iv) optionally comparing the activity determined in step (iii) with SK-1 and/or SK-2 channel opening activity following incubation of a cell, preferably said cell, in the presence of said test agent thereby determining the selectivity of said test agent for SK3 channels;

(v) providing a chemically modified version of said agent of step (iii) or (iv);

(vi) administrating said version of step (v) to a heathly, human volunteer not afflicted with bipolar disorder.

A SK-3 channel opener typically alters the firing rate or firing pattern of dopamine neurons in the ventral tegmental area of the brain. The SK-3 channel opening activity of an agent may be determined by examining the activity of dopamine neurons in a rat or mouse brain following administration of the agent.

A manic state may be induced in an animal, for example a rat or a mouse, by administering amphetamine or a mixture of amphetamine and chlordiazepoxide. In an animal model of mania the firing of dopamine neurons in the ventral tegmental area will typically be increased. The effect of a SK-3 channel opener on the activity of dopamine neurons in the ventral tegmental area may be monitored using such an animal model. The effect of a SK-3 channel opener on the behavioural changes, such as locomotor changes, observed in an animal model of mania may also be monitored. A SK-3 channel opener suitable for use in treating bipolar disease will typically reduce the rate of neuronal firing and/or reduce abnormal behaviours such as those induced by administration of chlordiazepoxide and amphetamine.

An exemplary opener of SK-3 channels is 1-ethyl-2-benzimidazolinone (1-EIBO).

The present invention provides a method of treating a human patient afflicted with bipolar disorder, particularly the manic phase thereof, which method consists essentially of administering a therapeutically effective amount of a SK-3 channel opener to a patient in need of treatment. Such a method may involve self-administration.

Administration of a therapeutically effective amount of an SK-3 channel opener to a human patient afflicted with bipolar disorder will typically improve the condition of a patient afflicted with bipolar disorder and/or alleviate the symptoms of bipolar disorder. There are of course many other factors which will dictate overall clinical response to administration of a SK3 channel opener. These factors are matters for the attending physician.

Pharmaceutical preparations comprising an SK3 channel opener of the invention, also referred to herein as active ingredient(s), may be administered for therapy by any suitable route including oral, rectal, topical and parenteral (including subcutaneous, transdermal, intramuscular and intravenous). It will also be appreciated that the preferred route will vary with the conditions and age of the recipient and the chosen active ingredient.

In general, for the foregoing conditions a suitable dose of a SK3 channel opener of the invention is in the range of from 0.05 to 100 mg per kilogram of body weight of the receipient per day, preferably in the range of from 0.5 to 20 mg per kilogram body weight of the recipient per day and optimally from 1 to 10 mg per kilogram body weight per day. The desired dose is preferably presented as two, three, four, five, six or more sub-doses administered at appropriate intervals during the day. These sub-doses may be administered in unit dosage forms, for example, containing from 1 to 1500 mg, preferably from 5 to 1000 mg and most preferably from 10 to 700 mg of active ingredient per unit dosage form.

While it is possible for the active ingredient to be administered alone it is preferable to present it as a pharmaceutical preparation. The preparations of the present invention comprise at least one active ingredient as defined above together with one or more acceptable carriers therefor and optionally other therapeutic agents. Each carrier must be “acceptable” in the sense of being compatible with the other ingredients of the preparation and not injurious to the recipient. The SK3 channel opener may be formulated with standard carriers and/or excipients as is routine in the pharmaceutical art, and as fully described in Remington's Pharmaceutical Sciences, Mack Publishing Company, Eastern Pennsylvania 17^{th} Ed. 1985.

Preparations include those suitable for oral, rectal, topical or parenteral (including subcutaneous, intramuscular, transdermal and intravenous) administration. The preparations may conveniently be presented in unit dosage form and may be prepared by any methods well known in the art of pharmacy. Such methods include the step of bringing into association the active ingredient with the carrier which constitutes one or more accessory ingredients. In general the preparations are prepared by uniformly and intimately bringing into association the active ingredient with liquid carriers or finely divided solid carriers or both, and then if necessary shaping the product.

Preparations of the present invention suitable for oral administration may be presented as discreet units such as capsules, cachets or tablets each containing a predetermined amound of the active ingredient; as powder or granules; as a solution or suspension in an aqueous or non-aqueous liquid; or as an oil-in-water liquid emulsion or in a water-in-oil liquid emulsion. The active ingredient may also be presented as a bolus, electuary or paste.

A tablet may be made by compression or moulding, optionally with one or more accessory ingredients. Compressed tablets may be prepared by compressing in a suitable machine the active ingredient in a free-flowing form such as a powder or granules, optionally mixed with a binder (e.g. povidone, gelatin, hydroxypropylmethyl cellulose), lubricant, inert diluent, preservative, idsintegrant (e.g. sodium starch glycollate, cross-lined povidone, cross-linked sodium carboxymethyl cellulose) surface-active or dispersing agent. Moulded tablets may be made by moulding in a suitable machine a mixture of powdered compound moistened with an inert liquid diluent. The tablets may be optionally coated or scored and may be formulated so as to provide slow or controlled release of the active ingredient therein using, for example, hydroxypropylmethyl cellulose in varying proportions to provide the desired release profile. Tablets may optionally be provided with an enteric coating, to provide release in parts of the gut other than the stomach.

Preparations suitable for oral use as described above may also include buffering agents designed to neutralise stomach acidity. Such buffers may be chosen from a variety of organic or inorganic agents such as weak acids or bases admixed with their conjugated salts.

Preparations for rectal administration may be presented as a suppository with a suitable base comprising, for example, cocoa butter or a salicylate.

Preparations suitable for parenteral administration include aqueous and non-aqueous sterile injection solutions which may contain anti-oxidants, buffers, bacteriostats and solutes which render the preparation isotonic with the blood of the intended recipient; and aqueous and non-aqueous sterile suspensions which may include suspending agents and thickening agents, as liposomes or other microparticulate systems which are designed to target the compounds to one or more organs. The preparation may be presented in unit-dose or multi-dose sealed containers, for example ampoules or vials and, and may be stored in a freeze dried (lyophilised) condition requiring only the addition of the sterile liquid carrier, for example water for injections, immediately prior to use. Extemporaneous injection solutions and suspensions may be prepared from sterile powders, granules and tablets of the kind previously described.

Preparations suitable for transdermal administration may be presented as discreet patches adapted to remain in intimate contact with the epidermis of the recipient for a prolonged period of time. Such patches typically contain the active ingredient as an optionally buffered aqueous solution of, for example, from 0.1 to 0.2M concentration with respect to the said active ingredient. As one particular possibility, the active ingredient, may be delivered from the patch by iontophoresis as generally described in Pharmaceutical Research 3(6), 318(1986).

Preferred unit dosage preparations are those containing a daily dose or unit, daily sub-dose, as herein above recited, or an appropriate fraction thereof, of an active ingredient.

It should be understood that in addition to the ingredients particularly mentioned above the preparations of this invention may include other agents conventional in the art having regard to the type of preparation in question, for example, those suitable for oral administration may include such further agents as sweetners, thickeners and flavouring agents.

Combination therapy in which the medicaments of the present invention are used simultaneously, sequentially or separately with other different therapeutic agents for the treatment of bipolar disorder, particularly the manic phase are also envisaged.

### Exemplification

The present invention is described by way of example only and with reference to the following figure in which:

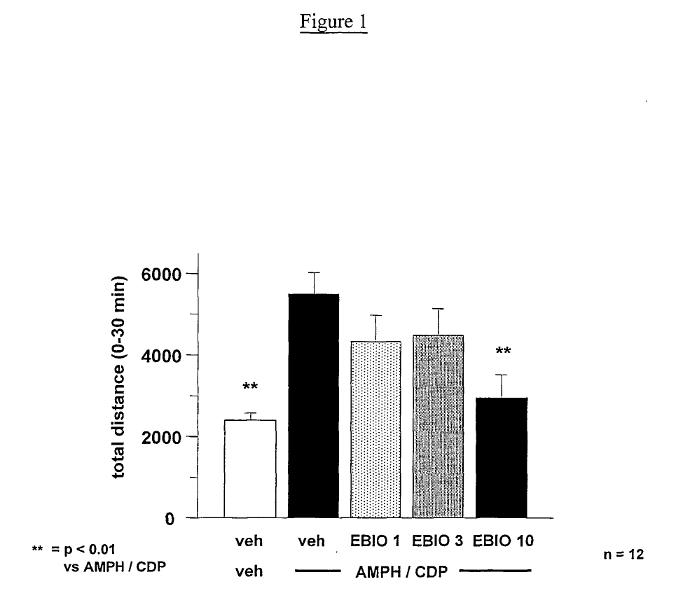

FIG. 1: The reduction by EBIO of locomotor activity in mice (n=12) induced by the administration of amphetamine (AMPH) and chlordiazepoxide (CDP). All compounds were administered 30 minutes before measurement of locomotor activity. The graph shows the mean and standard error of the mean of the total distance travelled for mice in each treatment group.

### EXAMPLE 1

### Cloning of hSK-3 Channel

The full-length hSK-3 clone was obtained from Incyte database (Incyte Pharmaceuticals, Inc.) and originated from a human prostate library (Genbank assession number AJ 251016). The coding region from this clone was obtained by PCR. An optimal Kozak sequence (Kozak, 1987) was added to the initiation site and the gene was inserted into the pCIN5 vector (Rees et al., 1996). By this insertion the hSK-3 gene was located downstream of a CMV promoter and upstream of an IRES box extending translation to a following Neo^{r} gene. After amplification in E. Coli, plasmid DNA was prepared and purified using QIAGEN Tip 500 Kit according to the manufacturer's instructions.

### EXAMPLE 2

### Transfection and Chemical Clone Selection

Five μg of the purified plasmid DNA was used to transfect 5×10⁶ CHO-K1 cells (ATCC N^{o} CRL 9618) by electroporation (standard protocol with BIORAD Gene Pulser II). After two days of growth in Petri dishes in alpha-MEM (Gibco BRL, Cat. No. 22571) and 10% FBS (Gibco BRL, Cat. No. 10108-165), chemical selection was initiated by addition of the antibiotic G418-sulfate (Calbiochem) at a final concentration of 0.5 mg/ml. After another two weeks of culture, 47 antibiotic-resistant clones were isolated, expanded under antibiotic selection and subsequently stored frozen in liquid nitrogen until further analysis.

### EXAMPLE 3

### Functional Clone Selection and Cell Culture

In order to select clones which express hSK-3 channel in a functionally correct manner, a fluoresence assay using the membrane potential-sensitive fluorescence dye DiBAC₄(3) was utilised (see below). Out of 47 clones (see above), the one which showed highest activation after increase in intracellular calcium concentration was expanded, cultured under antibiotic selection (0.5 mg/ml G418 sulfate) and stored frozen in liquid nitrogen. CHO-K1 cells were cultured using Alpha-MEM with 10% heat inactivated foetal bovine serum (Gibco BRL) at 37° C. and 10% CO₂.

### EXAMPLE 4

### Indentification of SK-3 Channel Openers Using a Dibac₄(3) Fluorescence Assay

Screening of test compounds for SK-3 channel opening activity is carried out using the membrane potential-sensitive fluorescence dye DiBACK₄(3). The assay is carried out using recombinant CHO cells cultured in black 96-well microplates with a clear bottom (Costar Cat. No. 3603). After 2-3 days of culture, cell culture medium is replaced with assay buffer (1 mM KCl, 2.3 mM CaCl₂, 5 mM NaHCO₃, 1 mM MgCl₂, 154 mM NaCl, 5.5 mM D(+)-glucose, 5 mM HEPES, pH 7.4) containing 5 μM DiBAC₄(3). After 30 minutes of cell loading with the dye at 37° C., fluoresence (exicitation 488 nm, emission 510-560 nm) is read through the bottom of the microplate using a fluorescence plate reader (e.g. FLIPR, Molecular Devices). Activation of SK-3 channels is monitored by reading the fluorescence after adding compounds to be analysed for their channel modulating properties.

### EXAMPLE 5

### The Effects of the SK3 Channel Opener, EBIO in a Rodent Model of Mania

A model of mania in rodents has been reported in the literature (Cao and Peng, 1993; Serpa and Meltzer, 1999). In this model, mice are dosed with a mixture of amphetamine and chlordiazepoxide. This mixture induces a behavioural response that is considered to be different from that induced by amphetamine alone, and is suggested to model manic behaviour observed in humans with bipolar disorder. Previous validation studies have demonstrated that the behaviour induced by amphetamine plus chlordiazepoxide can be ameliorated by two drugs, lithium and sodium valproate, which are used for the treatment of mania in humans.

The methods used were as follows. Male CD1 mice (22-26 g from Charles River, Italy) were treated with amphetamine sulphate (1.25 mg/kg) and chlordiazepoxide hydrochloride (6.25 mg/kg). Drugs were dissolved in saline and administered via the intraperitoneal route, in a volume of 10 ml/kg, 30 minutes before the test session. During the test session, the locomotor activity of the mice was recorded using a Digiscan Analyzer (Omnitech, Model RXYZCM-8). Briefly, animals were individually placed in Plexiglas cages equipped with 48 photocells and the total distance travelled by each mouse over a 30 minute period was determined.

In the current study, we examined the effect of the SK channel opener, 1-ethyl-2-benzimidazolinone (EBIO), which is known to activate SK3 channels (Xia et al, 1998; Syme et al, 2000), in the model. The results show that EBIO can reduce hyperactivity in mice induced by a mixture of amphetamine and chlordiazepoxide (FIG. 1). We have also demonstrated, in agreement with the literature reports, that amphetamine/chlordiazepoxide-induced hyperactivity can be prevented by treatment with the mood stabiliser, sodium valproate at a dose of 150 mg/kg i.p. in mice (data not shown).

These results suggest that compounds that open SK3 channels may be useful in the treatment of mania in patients with bipolar disorder.

References. The entire contents of the following documents are incorporated herein by reference.

Cao, B-J. and Peng, N-A. (1993) Eur.J.Pharm. 237,177-181.

Serpa, K. A., and Meltzer, L. T. (1999) Society for Neuroscience Abstracts 25, 533.13.

Syme, C. A., Gerlach, A. C., Singh, A. K., Devor, D. C. (2000) Am.J.Physiol.Cell Physiol. 278, C570-C581.

Xia, X. M., Fakler, B., Rivard, A., Wayman, G., Johnson-Pais, T., Keen, J. E., Ishii, T., Hirschberg, B., Bond, C. T., Lutsenko, S., Maylie, J., Adelman, J. P. (1998) Nature, 395, 503-507.

1. Use of a SK-3 channel opener in the manufacture of a medicament for the treatment of bipolar disorder.

2. Use according to claim 1 wherein the SK-3 channel opener is a specific SK-3 channel opener.

3. Use according to claim 1 or 2 wherein the said medicament is for the treatment of the manic phase of bipolar disorder.

4. Use according to any preceding claim wherein said SK-3 channel opener is 1-ethyl-2-benzimidazolinone.

5. A method of treating a patient suffering from bipolar disorder, which method commprises administering a therapeutically effective amount of a compound of an SK-3 channel opener to a patient in need thereof.

6. A method according to claim 5 wherein the said SK-3 channel opener is a specific SK-3 channel opener.

7. A method of treating a patient afflicted with bipolar disorder, which method comprises the steps of:
(i) identifying a specific SK-3 channel opener; and
(ii) administering a therapeutically effective amount of the said SK-3 channel opener of step (I) to a patient in need thereof.

8. A method of identifying a lead series of candidate agents for the treatment of bipolar disorder, particularly the manic phase thereof, which method comprises the steps of;
(v) expressing a recombinant SK-3 channel in a cell;
(vi) incubating said cell in the presence of a test agent;
(vii) assessing SK-3 channel activity;
thereby determining whether the test agent acts as an opener of SK3 channels;
(iii) optionally comparing the activity determined in step (iii) with SK-1 and/or SK-2 channel opening activity following incubation of a cell, preferably said cell, in the presence of said test agent thereby determining the selectivity of said test agent for SK3 channels.

9. A method of identifying a SK3 channel opener for the treatment of bipolar disorder, which method comprises;
(i) expressing a recombinant SK-3 channel in a cell;
(ii) incubating said cell in the presence of a test agent;
(iii) assessing SK-3 channel activity
thereby determining whether the test agent acts as an opener of SK-3 channels;
(iv) optionally comparing the activity determined in step (iii) with SK-1 and/or SK-2 channel opening activity following incubation of a cell expressing a SK1 and/or SK2 channel, preferably said cell, in the presence of said test thereby determining the selectivity of said test agent for SK3 channels.

10. A method of developing a SK3 channel opener suitable for administration to a human patient afflicted with bipolar disorder, particularly for the manic phase thereof, which method comprises;
(i) expressing a recombinant SK-3 channel in a cell;
(ii) incubating said cell in the presence of a test agent;
(iii) assessing SK-3 channel activity;
thereby determining whether the test agent acts as an opener of SK-3 channels;
(iv) optionally comparing the activity determined in step (iii) with SK-1 and/or SK-2 channel opening activity following incubation of a cell in the presence of said test agent thereby determining the selectivity of said test agent for SK3 channels;
(v) providing a chemically modified version of said agent of step (iii) or (iv).
(vi) Optionally administrating said version of step (v) to a human patient or volunteer afflicted with bipolar disorder;
(vii) assessing whether the condition and/or symptoms of said patient or volunteer of step (vi) improves.

11. A method of developing a SK3 channel opener suitable for administration to a human patient afflicted with bipolar disorder, particularly for the manic phase thereof, which method comprises;
(i) expressing a recombinant SK-3 channel in a cell;
(ii) incubating said cell in the presence of a test agent;
(iii) assessing SK-3 channel activity;
thereby determining whether the test agent acts as an opener of SK3 channels;
(iv) optionally comparing the activity determined in step (iii) with SK-1 and/or SK-2 channel opening activity following incubation of a cell in the presence of said test agent thereby determining the selectivity of said test agent for SK3 channels;
(v) providing a chemically modified version of said agent of step (iii) or (iv);
(vi) using said version of step (v) in the manufacture of a medicament.
